(19) United States
(12) Patent Application Publication
(10) Pub. No.: US 2007/0093759 A1
(43) Pub. Date: Apr. 26, 2007

(54) HIGH VISCOSITY MATERIAL DELIVERY SYSTEM

(75) Inventor: Michael F. Sickler, Laguna Hills, CA (US)

Correspondence Address: BANNER & WITCOFF, LTD., 28 STATE STREET, 28th FLOOR, BOSTON, MA, 02109-9601, US

(73) Assignee: IsoTis OrthoBiologics, Inc., Irvine, CA (US)

(21) Appl. No.: 11/535,265

(22) Filed: Sep. 26, 2006

Related U.S. Application Data
(60) Provisional application No. 60/720,586, filed on Sep. 26, 2005.

(51) Int. Cl. A61M 5/00

(52) U.S. Cl. 604/181

(57) ABSTRACT

A delivery system for high viscosity material includes a cannula having an axis, an opening at a first end thereof, and an opening at a second end thereof. A dispenser includes a body member having a reservoir, a first opening at a first end thereof in fluid communication with the first end of the cannula; and a second opening at a second end thereof. A first transfer member has an aperture extending axially therethrough and is slidably received in the reservoir of the body member. A second transfer member has an aperture extending axially therethrough and is slidably received in the aperture of the first transfer member. A third transfer member is slidably received in the aperture in the second transfer member.

### RELATED APPLICATION(S)

This application claims priority to provisional U.S. Application Ser. No. 60/720,586, filed Sep. 26, 2005.

### FIELD OF THE INVENTION

Described herein are delivery devices suitable for introduction of high viscosity materials into the body. More specifically, the described delivery systems are particularly suitable for the delivery of high viscosity materials into constricted areas, and as such, are particularly useful in surgical procedures.

### BACKGROUND OF THE INVENTION

Tissue regeneration materials may be used to fill bone defects to effect bone grafts. Since the tissue regeneration materials often have high viscosity, they are difficult to deliver to a surgical site using conventional delivery devices such as syringes. High viscosity materials are difficult to force out of a conventional syringe fitted with a needle. As a practical matter, conventional syringes when used alone (i.e., without a needle) are usually too large or too short for insertion into a desired treatment area. Smaller (or “down-sized”) syringes are often difficult to control when applying the high forces necessary to press viscous materials through the small exit bore. Furthermore, conventional syringes, whether used with or without needles, may be inefficient in that they often retain a portion of the tissue regeneration material inside the syringe body or needle.

It would be desirable to provide a device that is able to deliver a high viscosity material to a constricted area with good control, and to reduce the amount of wasted or undeliverable material that would otherwise remain within the delivery device.

Particular objects and advantages will be apparent to those skilled in the art, that is, those who are knowledgeable or experienced in this field of technology, in view of the following disclosure of the invention and detailed description of certain embodiments.

### SUMMARY

Described here is a delivery system suitable for delivering a high viscosity material to a constricted area. An example of such a use or procedure is the delivery of a tissue regeneration material into a graft site, or the like. When used properly, the system can reduce the amount of material that would otherwise remain within the delivery system.

Certain embodiments of the delivery system, due to their design, are easy to manipulate and to control when dispensing high viscosity material. The system design may also be configured to be low cost and perhaps disposable. This is an advantage when prevention of cross-contamination or a desire for avoiding “clean up” is a significant issue with respect to design parameters.

Also described is a method of using the minimally invasive high viscosity material delivery system.

In accordance with a first aspect, a delivery system for high viscosity material includes a cannula having an axis, an opening at a first end thereof, and an opening at a second end thereof. A dispenser includes a body member having a reservoir, a first opening at a first end thereof in fluid communication with the first end of the cannula; and a second opening at a second end thereof. A first transfer member has an aperture extending axially therethrough and is slidably received in the reservoir of the body member. A second transfer member has an aperture extending axially therethrough and is slidably received in the aperture of the first transfer member. A third transfer member is slidably received in the aperture in the second transfer member.

In accordance with another aspect, a delivery system for high viscosity material includes a cannula having an axis, an opening at a first end thereof, and an opening at a second end thereof. A dispenser includes a body member having a reservoir, a first opening at a first end thereof; a second opening at a second end thereof, and a handle at the second end, with the first end being secured to second end of the cannula. A first transfer member has an aperture extending axially therethrough and a handle, with the first transfer member being slidably received in the reservoir of the body member. A second transfer member has an aperture extending axially therethrough and is slidably received in the aperture of the first transfer member. A third transfer member has a handle and is slidably received in the aperture in the second transfer member.

In accordance with a further aspect, a dispenser includes a body member having a reservoir formed therein, a first opening at a first end thereof; a second opening at a second end thereof, and a handle at the second end. A first transfer member has an aperture extending axially therethrough, a seal at a first end thereof and a handle at a second end thereof, with the first transfer member being slidably received in the reservoir of the body member. A second transfer member has an aperture extending axially therethrough, a seal at one end thereof, and is slidably received in the aperture of the first transfer member. A third transfer member has a handle and is slidably received in the aperture in the second transfer member. A cannula has an opening at a first end thereof, and an opening at a second end thereof, with the second end being releasably secured to the first end of the body member.

These and additional features and advantages disclosed here will be further understood from the following detailed disclosure of certain embodiments.

### BRIEF DESCRIPTION OF THE DRAWINGS

FIG. 1 is a perspective view, in exploded form, of a high viscosity material delivery system.

FIG. 2 is a section view of the high viscosity material delivery system of FIG. 1, shown in an assembled fashion.

FIG. 3 is a perspective view of the high viscosity material delivery system of FIG. 1 in a partially assembled fashion.

FIG. 4 is a perspective view of one embodiment of a cannula for use with the high viscosity material delivery system of FIG. 1.

FIG. 5 is a perspective view of another embodiment of a cannula for use with the high viscosity material delivery system of FIG. 1.

FIG. 6 is a perspective view, in exploded form, of another high viscosity material delivery system.

FIG. 7 is a section view of the high viscosity material delivery system of FIG. 6, shown in an assembled fashion.

FIG. 8 is a perspective view of the high viscosity material delivery system of FIG. 6 in a partially assembled fashion.

FIG. 9 is a perspective view of one embodiment of a cannula for use with the high viscosity material delivery system of FIG. 6.

FIG. 10 is a perspective view of another embodiment of a cannula for use with the high viscosity material delivery system of FIG. 6.

The figures referred to above are not drawn necessarily to scale and should be understood to provide a representation of the invention, illustrative of the principles involved. Some features of the high viscosity material delivery system depicted in the drawings have been enlarged or distorted relative to others to facilitate explanation and understanding. The same reference numbers are used in the drawings for similar or identical components and features shown in various alternative embodiments. High viscosity material delivery systems as disclosed herein would have configurations and components determined, in part, by the intended application and environment in which they are used.

### DETAILED DESCRIPTION OF CERTAIN PREFERRED EMBODIMENTS

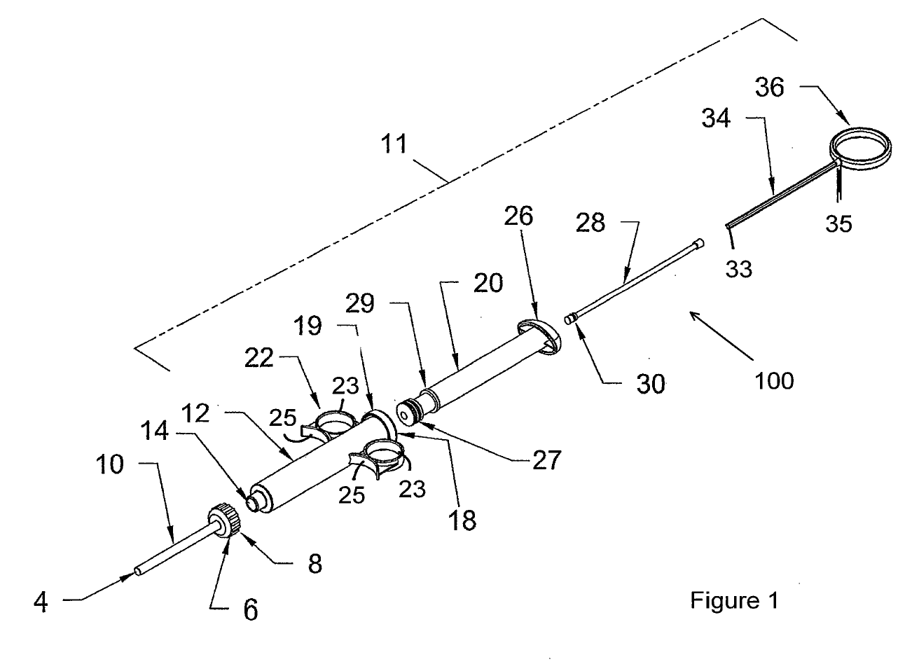

FIGS. 1-3 show a delivery system 100 for placement of a high viscosity material. In certain embodiments, delivery system 100 is suitable for placement of a high viscosity material in a mammal. Delivery system 100 may be used for placement of a high viscosity material into a human body, for example. Delivery system 100 includes a cannula 10 and a dispenser 11 that dispenses a high viscosity material from a first open delivery end of cannula 10, that defines an opening or orifice 4, to a desired treatment site. The high viscosity material may be a gel, putty, paste, flowable composition containing particulates, high viscosity liquid (e.g., a liquid that is more viscous than water or the like), any combination thereof, or the like. A second end 6 of the cannula 10 opposite the delivery end 4 is associated with the dispenser 11.

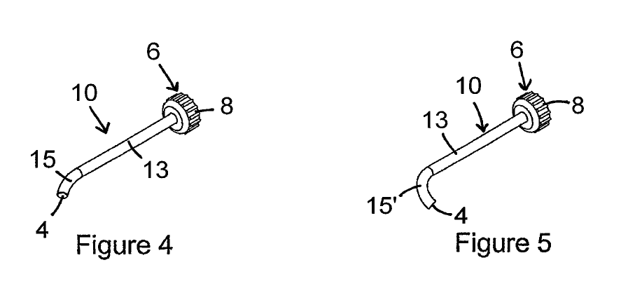

In certain embodiments, second end 6 of cannula 10 is connected to dispenser 11 by means of internal threads. An exemplary embodiment of this connection as shown in FIGS. 1-3 includes an internally threaded adapter 8 on second end 6 and external threads 9 on dispenser 11. Dispenser 11 is positioned so that it presses the high viscosity material 2 through and out of cannula 10. Cannula 10 can be designed in any suitable shape for delivery such as flexible, straight, bent, curved, or the like. Two exemplary designs of the cannula 10 are shown in FIGS. 4-5. In the embodiment illustrated in FIG. 4, cannula 10 has a straight first portion 13 and a second portion 15 at its tip that is slightly curved. In the embodiment illustrated in FIG. 5, cannula 10 has a second portion 15′ with a much greater curve. In the illustrated embodiment, second portion 15′ curves in an arc extending approximately 90°. The amount of curvature of second portions 15, 15′ can be varied, and suitable amounts of curvature will be dependent upon the application and environment in which cannula 10 is used. Suitable amounts of curvature will become readily apparent to those skilled in the art, given the benefit of this disclosure.

Cannula 10 may be constructed of a suitable material, e.g., metal, metallic alloys, plastics, glass, plastic composites (e.g., ABS and Barium Sulfate) or the like, capable of providing the strength needed to safely introduce a high viscosity material into a treatment site. In certain embodiments, stainless steel, polycarbonate, polypropylene, polyethylene, PTFE (Teflon), and ABS have been found to be suitable materials for cannula 10.

The length and width and wall thickness of cannula 10 may be varied depending, in general, upon the desired application. For instance, the length, inner diameter, and outer diameter of the cannula may be chosen to be, respectively, in the range of 5-35 cm, 1-20 mm, and 2-25 mm; or 7-30 cm, 1-15 mm, and 2-20 mm; or 10-25 cm, 1-6 mm, and 2-10 mm.

Dispenser 11 provides pressure to the high viscosity material in the cannula 10 causing axial movement of the high viscosity material through cannula 10, and metered delivery of the high viscosity material 2 through its first, delivery end 4. The dispensing pressure may be applied in any art-disclosed ways (e.g., manually or pneumatically, actuated plungers; or the like) that is adapted for axial displacement of the high viscosity material positioned within cannula 10.

Dispenser 11 includes a body member 12 having a first opening 14 at a first end thereof that is in fluid communication with cannula 10. The first end of body member 12 is releasably secured to second end 6 of cannula 10.

Body member 12 includes a reservoir 16 for containing high viscosity material, a second opening 18 associated with a first transfer member 20, and a handle 22. In the illustrated embodiment, handle 22 is formed of a pair of rings 23 secured to an exterior of body member 12, spaced approximately 180° from one another about the circumference of body member 12. An arcuate surface 25 is formed on an exterior of each ring 23 as an alternative position for the fingers of the user.

A first transfer member 20 is slidably received in reservoir 16 and serves to force the high viscosity material out of reservoir 16. Body member 12 may also include a retainer cap 19 for retaining and preventing first transfer member 20 from sliding out of body member 12 via second opening 14. First transfer member 20 is received in second opening 18 of body member 12. When pressure is applied to first transfer member 20, it moves the high viscosity material axially along reservoir 16, allowing the transfer of the high viscosity material from reservoir 16 into cannula 10 via first opening 14, and then from cannula 10 to the desired treatment site via its open delivery end 4.

Body member 12 may be constructed of a suitable material, e.g., metal, metallic alloys, plastics, glass, or the like, capable of withstanding the substantial pressures generated during use.

First transfer member 20 has an aperture 24 extending therethrough, providing an interior passageway axially along first transfer member 20. In certain embodiments, a seal 27 is provided on a first end of first transfer member 20 and serves to provide a sterile barrier for the high viscosity material located within reservoir 16 when the first end of first transfer member 20 is positioned within body member 12 by engaging the interior surface of reservoir 16. A handle 26 in the form of a cap is provided on a second end of first transfer member.

In certain embodiments, seal 27 may be a wiper and pressure seal and, as such, wipes reservoir 16 of the high viscosity material as first transfer member 20 moves axially within reservoir 16. Seal 27 serves to maintains the pressure on the high viscosity material forward of first transfer member 20 as it moves within reservoir 16 toward cannula 10. Seal 27 can be constructed using any suitable art-disclosed means. For example, as shown in FIG. 1, seal 27 may be constructed of dual o-rings seated in a flange formed at the first end of first transfer member 20.

First transfer member 20 may include a stop 29 in the form of a flange that works in conjunction with retainer cap 19 to prevent first transfer member 20 from sliding out of body member 12 via the second opening 18.

Dispenser 11 also includes a second transfer member 28 that is slidably received in aperture 24 of first transfer member 20. As shown in FIG. 1, second transfer member 28 may have a seal 30 on a first end thereof that engages the surface of aperture 24 to provide a sterile barrier for the high viscosity material in reservoir 16 and with which second transfer member 28 is in communication. Seal 30 may be a wiper and pressure seal and, as such, wipes aperture hole 24 clean of the high viscosity material as second transfer member moves axially through aperture 24. Seal 30 serves to maintain pressure on the high viscosity material forward of second transfer member 28 as it moves though aperture 24. Seal 30 helps to prevent any high viscosity material from remaining inside aperture 24.

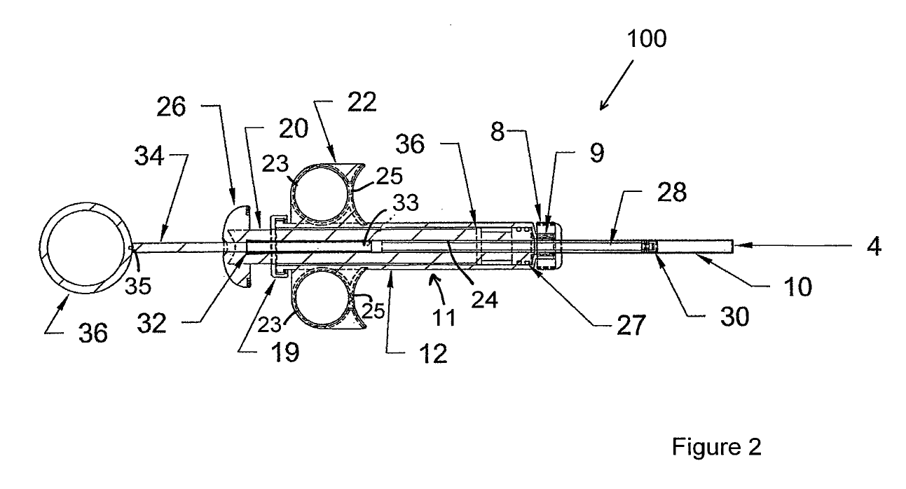

Seal 30 can be constructed using suitable art-disclosed means such as o-ring, silicon material, or the like. As shown in FIG. 1, seal 30 is formed of an o-ring seated in a flange at the first end of second transfer member 26. As shown in FIG. 2, first transfer member 20 may have a shoulder or internal stop 32 that prevents second transfer member 28 from backtracking or sliding back out of first transfer member 20. Internal stop 32 can be constructed using any suitable art-disclosed means.

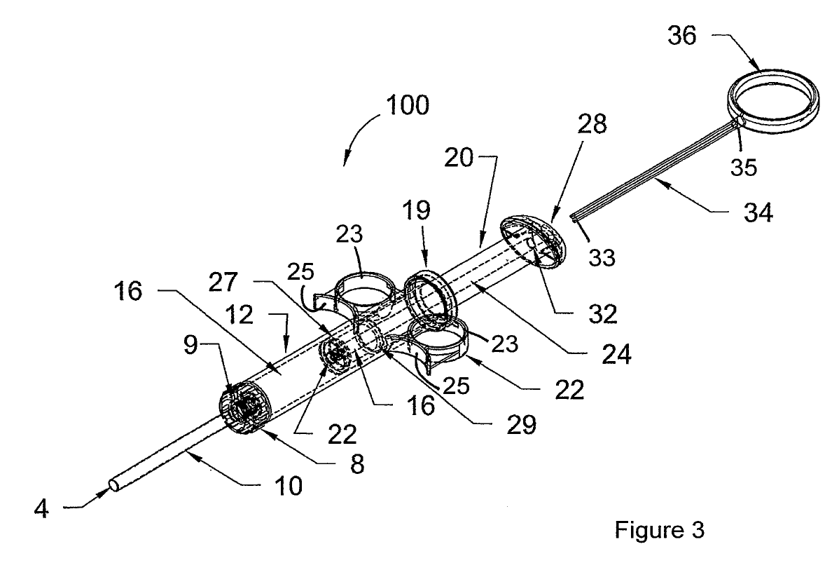

Dispenser 11 further includes a third transfer member 34 having a first end 33 and a handle 36 at its second end 35. In certain embodiments, as seen in FIG. 3, third transfer member 34 may have a substantially X shaped cross-section. It is to be appreciated that third transfer member 34 may have any desired cross-sectional shape.

In certain embodiments, handle 36 may be a circular ring as shown here. It is to be appreciated that handle 36 can have any desired shape. Third transfer member 34 is slidably received in aperture 24 in first transfer member 20 and acts like a piston by pushing second transfer member 28 through aperture 24, thereby causing second transfer member 28 to displace and expel the high viscosity material 2 that is found in aperture 24 and cannula 10 through open delivery end 4 of cannula 10. The combination of first transfer member 20, second transfer member 28, and third transfer member 34 serves to deliver of all of the high viscosity material that is located within reservoir 16 and cannula 10 to the desired treatment site.

Each of first transfer member 20, second transfer member 28, third transfer member 24, handles 22, 26, and 36, stop 29, and internal stop 32 may be constructed of a suitable material such as metal (e.g., aluminum or the like), metallic alloys (e.g., stainless steel or the like), plastics (e.g., PVC, polycarbonate, ABS, acrylic, or the like), silicon, or the like. In certain embodiments second transfer member 28 may be constructed of a flexible material, which is advantageous when used with a cannula 10 having a portion shaped in a non-linear fashion, such as the curved tips seen in FIGS. 4-5. A flexible second transfer member 28 can easily enter the interior passageway of such a non-linear cannula 10.

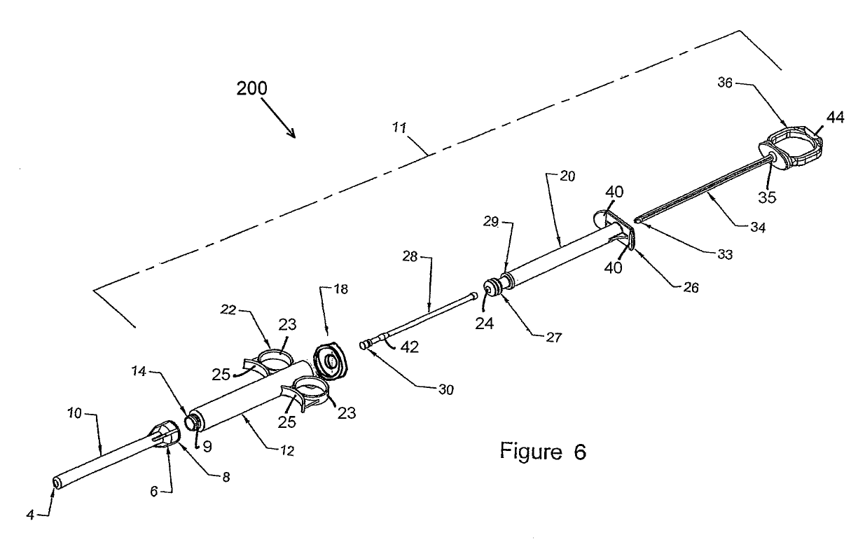

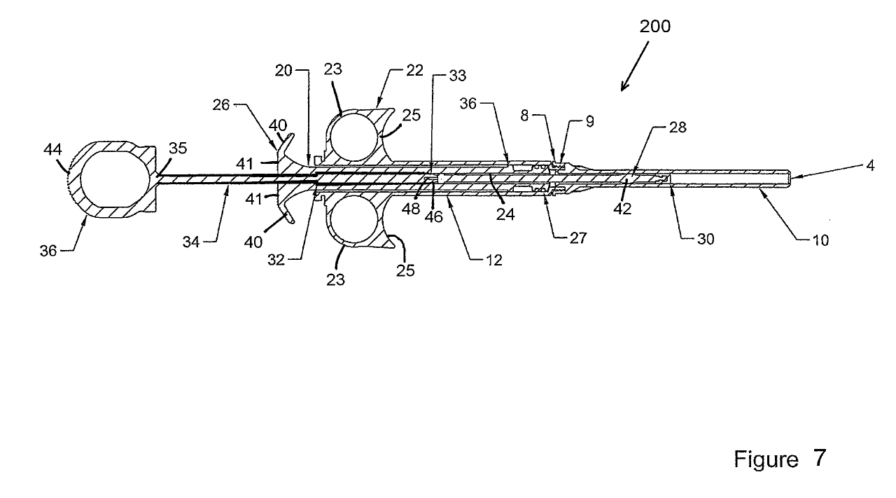

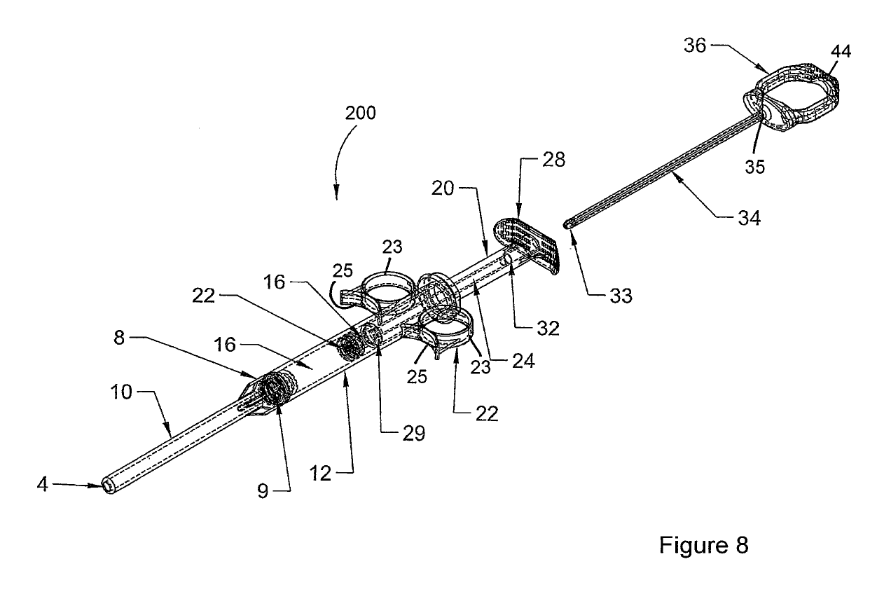

A second embodiment of a delivery system 200 is shown in FIGS. 6-10. In this embodiment, handle 26 on first transfer member 20 is in the form of a pair of flanges 40 extending outwardly from the second end of first transfer member 20, spaced approximately 180° from one another about the circumference of first transfer member 2.

Serrations 41 are provided on flanges 40 to enhance the grip of the user's fingers.

A raised rib 42 is formed on second transfer member 28, proximate its first end. Raised rib 42 serves to reduce the chance of second transfer member 28 being pulled out of aperture 24 of first transfer member 20. Serrations 44 are formed on the end of handle 36 to prevent slippage when the user's fingers are dispensing high viscosity material through delivery system 200. First end 33 of third transfer member is bifurcated to define a pair of barbs 46 with a gap 48 positioned therebetween. The bifurcated first end 33 engages internal stop 32 in first transfer member 20 in snap-fit fashion.

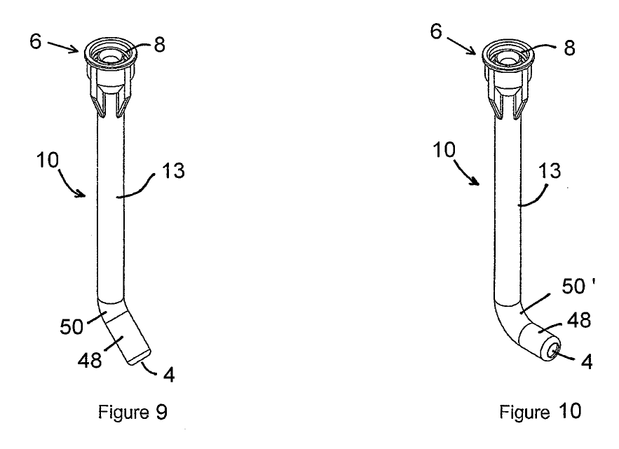

As seen in FIGS. 9-10, cannula 10 may be curved or non-linear. As seen in FIG. 9, cannula 10 has a first linear portion 13 connected to a second linear portion 48 by way of a curved connecting portion 50. In the illustrated embodiment, curved connecting portion 50 is curved about an angle of approximately 45°.

As seen in FIG. 10, first linear portion 13 is connected to second linear portion 48 by way of a curved connecting portion 50′ that curves about an angle of approximately 90°. It is to be appreciated that the curved connecting portion of cannula 10 can curve about any desired angle, thereby orienting open end 4 at any orientation with respect to body 12.

To prevent cross-contamination and need for clean up, the entire delivery system 100 may be constructed of inexpensive, disposable materials and be disposed of when reservoir 16 is depleted. Alternatively, delivery system 100 may be cleaned and reused. If reuse is desired, delivery device 100 may be constructed of materials that are autoclavable. Regardless of whether delivery system 100 is disposable or autoclavable, each part of delivery system 100 that comes into contact with the high viscosity material may be formed of a material that is chemically inert to the high viscosity material.

The accompanying figures and this description depict variations of the described high viscosity material delivery system and its components. Conventional fasteners such as snap fits, rivets, machine screws, nut and bolt connectors, machine threaded connectors, snap rings, clamps, toggles, pins, and the like may be used to connect the various components. Friction fitting, welding, or deformation, if suitable may be used as appropriate to connect the various components. Furthermore, materials for making the components of the system, unless otherwise specified, may be selected from appropriate materials such as metals, metallic alloys, fibers, plastics, and the like. Appropriate production methods may include casting, extruding, molding, machining, or the like.

The described system may be used to conduct a method for delivering high viscosity material by providing a high viscosity material delivery system 100 as described above; placing high viscosity material into reservoir 16; and transferring the high viscosity material from reservoir 16 into cannula 10 via first opening 14 and dispensing the high viscosity material from cannula 10 by introducing pressure to cannula 10 from dispenser 11. The introduction of pressure to cannula 10 from dispenser 11 is achieved by pushing first transfer member 20 into reservoir 16 of body member 12 thereby forcing high viscosity material into cannula 10 and out through opening 4, inserting second transfer member 28 into aperture 24 of first transfer member 20, inserting third transfer member 34 into aperture 24 behind second transfer member 28, and pushing on third transfer member 34 to force second transfer member 28 through second aperture 24 and into cannula 10, thereby forcing any remaining high viscosity material out of cannula 10 into a desired treatment site.

In light of the foregoing disclosure of the invention and description of various embodiments, those skilled in this area of technology will readily understand that various modifications and adaptations can be made without departing from the scope and spirit of the invention. All such modifications and adaptations are intended to be covered by the following claims.

What is claimed is:

1. A delivery system for high viscosity material comprising:
a cannula having an axis, an opening at a first end thereof, and an opening at a second end thereof; and
a dispenser comprising:
a body member having a reservoir, a first opening at a first end thereof in fluid communication with the first end of the cannula; and a second opening at a second end thereof;
a first transfer member having an aperture extending axially therethrough and being slidably received in the reservoir of the body member;
a second transfer member having an aperture extending axially therethrough and being slidably received in the aperture of the first transfer member; and
a third transfer member slidably received in the aperture in the second transfer member.

2. The delivery system of claim 1, wherein the body member has a handle.

3. The delivery system of claim 2, wherein the handle comprises a pair of rings secured to an exterior surface of the body member.

4. The delivery system of claim 1, wherein the first transfer member has a handle.

5. The delivery system of claim 4, wherein the handle comprises a pair of flanges extending outwardly from an end of the first transfer member.

6. The delivery system of claim 1, wherein the third transfer member has a handle.

7. The delivery system of claim 1, wherein the handle comprises a ring secured to an end of the third transfer member.

8. The delivery system of claim 1, further comprising a seal at a first end of the first transfer member and configured to engage an interior surface of the reservoir.

9. The delivery system of claim 8, wherein the seal comprises an O-ring.

10. The delivery system of claim 1, further comprising a seal at a first end of the second transfer member.

11. The delivery system of claim 10, wherein the seal comprises an O-ring.

12. The delivery system of claim 1, further comprising threaded members to secure the first end of the body member to the second end of the cannula.

13. The delivery system of claim 12, wherein the threaded members comprise an internally threaded adapter on the second end of the cannula and external threads on the first end of the body member.

14. The delivery system of claim 1, wherein the cannula is non-linear.

15. The delivery system of claim 1, wherein the first end of the cannula is curved.

16. The delivery system of claim 1, wherein the second transfer member is formed of a flexible material.

17. A delivery system for high viscosity material comprising:
a cannula having an axis, an opening at a first end thereof, and an opening at a second end thereof; and
a dispenser comprising:
a body member having a reservoir, a first opening at a first end thereof; a second opening at a second end thereof, and a handle at the second end, the first end being secured to second end of the cannula;
a first transfer member having an aperture extending axially therethrough and a handle, the first transfer member being slidably received in the reservoir of the body member;
a second transfer member having an aperture extending axially therethrough and being slidably received in the aperture of the first transfer member; and
a third transfer member having a handle and being slidably received in the aperture in the second transfer member.

18. The delivery system of claim 17, further comprising a seal at a first end of the first transfer member and configured to engage an interior surface of the reservoir.

19. The delivery system of claim 18, wherein the seal comprises an O-ring.

20. The delivery system of claim 17, further comprising a seal at a first end of the second transfer member.

21. The delivery system of claim 20, wherein the seal comprises an O-ring.

22. A dispenser comprising:
a body member having a reservoir formed therein, a first opening at a first end thereof; a second opening at a second end thereof, and a handle at the second end;
a first transfer member having an aperture extending axially therethrough, a seal at a first end thereof and a handle at a second end thereof, the first transfer member being slidably received in the reservoir of the body member;
a second transfer member having an aperture extending axially therethrough, a seal at one end thereof, and being slidably received in the aperture of the first transfer member;
a third transfer member having a handle and being slidably received in the aperture in the second transfer member; and
a cannula having an opening at a first end thereof, and an opening at a second end thereof, the second end being releasably secured to the first end of the body member.

23. The dispenser of claim 22, wherein each of the seal at the first end of the first transfer member and the seal of the second transfer member is an o-ring.
